(19) United States
(12) Patent Application Publication
(10) Pub. No.: US 2012/0279514 A1
(43) Pub. Date: Nov. 8, 2012

(54) COSMETIC KIT FOR MAKING UP AND/OR FOR CARE OF KERATINOUS MATERIAL

(75) Inventors: Audrey Ricard, La Varenne (FR); Sophie Bodelin, Vanves (FR)

(73) Assignee: L'OREAL, Paris (FR)

(21) Appl. No.: 13/393,772

(22) PCT Filed: Sep. 10, 2010
(86) PCT No.: PCT/IB2010/054090

§ 371 Date: Jul. 19, 2012

Related U.S. Application Data
(60) Provisional application No. 61/272,374, filed on Sep. 17, 2009.

(30) Foreign Application Priority Data
Sep. 11, 2009 (FR) 0956270

(51) Int. Cl. A45D 34/04; A61K 8/06; A61Q 1/02; A45D 40/26

(52) U.S. Cl. 132/200; 132/317; 132/318

(57) ABSTRACT

The present invention provides a cosmetic kit for making up and/or for care of keratinous material, the kit comprising at least: • a container containing at least one cosmetic composition in the form of a water-in-oil, oil-in-water or multiple emulsion, comprising at least 30% by weight of aqueous phase relative to the total weight of said composition and having a viscosity measured at 25° C. and at a shear rate of 200 min⁻¹ of strictly more than 0.8 Pa·s, said composition comprising at least one colorant, and • an applicator device (8) for applying the composition, provided with an applicator member (10) including an application surface that is capable of turning about at least one axis (X) or center of rotation in response to being moved in engagement with the keratinous material.

The present invention seeks to provide a novel mode for making up and/or for care of keratinous material and more particularly intended for application to a keratinous material, in particular to the skin, of fluid cosmetic compositions in the form of emulsions and including a high aqueous phase content.

Foundation compositions are routinely used to provide the skin, in particular the face, with an attractive color and also to camouflage skin imperfections such as blotches and/or spots. Many appropriate formulations have now been developed that can in particular be divided into those with a fluid structure and those with a solid structure.

More particularly, in the field of makeup and/or care compositions with a fluid texture, it is routine to use compositions in the form of water-in-oil (W/O) emulsions comprising an aqueous phase dispersed in a fatty phase. However, such emulsions may suffer from the disadvantage of being uncomfortable to apply, especially because of the greasy and heavy sensation afforded by the continuous fatty phase that can then give the user the sensation of applying a mask or of smothering.

In order to overcome those disadvantages, emulsions have been developed with a high hydroalcoholic phase content in order to provide the user with a sensation of freshness or a “fresh effect” on application of the composition to the keratinous material, in particular the skin.

As an example, application EP-A-1 068 851 proposes stable water-in-oil emulsions in the form of a cream having a high water content and including a particular silicone emulsifying agent.

Unfortunately, conventional applicator means, namely fingers or sponges, generally do not provide complete satisfaction, especially in terms of the sensory effect experienced by the user on application.

During application and smoothing of the composition with the fingers, the inherent heat of the fingers can affect the sensation of freshness that is desired.

Moreover, application with the fingers has the disadvantage of soiling the hands, which is clearly not desirable. That mode of application also causes problems as regards cleanliness, or even hygiene.

Further, the use of conventional means such as sponges, fingers or brushes do not in general result in a satisfactory makeup as regards uniformity of the deposit.

As a consequence, it would be desirable to have available a mode of making up that could enhance the feeling when applying compositions in the form of emulsions comprising a high aqueous phase content, in particular of encouraging the sensation of freshness, in particular over a long time, and also to improve the quality of the final makeup in particular in terms of uniformity of the deposit.

The present invention is precisely aimed at responding to this need.

More particularly, in exemplary embodiments the present invention provides a cosmetic kit for making up and/or for care of keratinous material, the kit comprising at least:
a container containing at least one cosmetic composition in the form of a water-in-oil, oil-in-water or multiple emulsion, comprising at least 30% by weight of aqueous phase relative to the total weight of said composition and having a viscosity measured at 25° C. and at a shear rate of 200 min⁻¹ [reciprocal minutes] of strictly more than 0.8 Pa·s; and
an applicator device for applying the composition, provided with an applicator member including an application surface that is capable of turning about at least one axis or center of rotation in response to being moved in engagement with the keratinous material.

In particular, a composition of the invention comprises at least one colorant, preferably at least one pigment.

In particular, a composition of the invention remains stable after at least two months storage at ambient temperature.

More particularly, a composition of the invention is fluid.

In the context of the present invention, the term “fluid” means a composition that is capable of flowing under its own weight, as opposed to compositions that are termed solid.

The composition may be a simple “water-in-oil” or “oil-in-water” type emulsion, or a multiple emulsion. Preferably, the composition is a water-in-oil emulsion.

In the expression “oil-in-water” or “water-in-oil”, the term “water” is used in a generic sense to mean an aqueous phase that may comprise water optionally mixed with at least one organic solvent. The aqueous phase is defined in more detail below.

In particularly preferred exemplary embodiments, the aqueous phase of a composition of the invention includes one or more lower C₂ to C₈ monoalcohols, in particular present in a quantity of at least 2% by weight relative to the total composition weight.

The fluid composition of the invention may be viscous to a greater or lesser extent, in the form of a lotion, or of a creamy or pasty appearance. It may also be in the form of a gel.

The composition of the invention may be a composition for making up and/or for care of the skin, in particular the face and/or the body, and may constitute a blusher, an eye shadow, a foundation, a concealer, a lipstick, a body makeup product, a face or body care product or a sunscreen.

More particularly, but not exclusively, the invention provides a foundation composition.

In a particular embodiment, a composition of the invention may have viscosity, measured at 25° C. and at a shear rate of 200 min⁻¹, of strictly more than 0.8 Pa·s and less than or equal to 4 Pa·s (pascal second], in particular in the range 0.9 Pa·s to 3.8 Pa·s and preferably in the range 1 Pa·s to 3.5 Pa·s.

Viscosity is measured at 25° C. with a CONTRAVES type TV viscosimeter provided with a No. 2 moving body, the measurement being carried out after 10 minutes rotation of the moving body (at the end of which, the viscosity and the rates of rotation of the moving body are observed to be stabilized), and at a shear rate of 200 min⁻¹.

In particular exemplary embodiments, the composition of the invention with the appearance of a cream in particular has a viscosity in the range 1 Pa·s. to 3.5 Pa·s., measured at 25° C. and at a shear rate of 200 min⁻¹.

The emulsion may have specific rheological characteristics that make it particularly advantageous in use. Thus, during application to the keratinous material, in particular the skin, at a pressure exerted by the applicator of the invention, the emulsion can “break”, i.e. it fluidizes abruptly under the effect of shear due to the movement of the applicator member over the skin, thereby releasing the aqueous phase and encouraging a sensation of great freshness.

In other particular exemplary embodiments, a composition of the invention may include at least one silicone surfactant, in particular selected from dimethicone polyols.

The applicator device is not limited to a single variant embodiment of the applicator member.

As can be seen from the accompanying figures, several variant embodimental embodiments may be envisaged provided that they are compatible with rotation of their application surface about at least one axis or center of rotation in response to said surface being moved in contact with the keratinous material to be treated, generally the skin.

The applicator member may be defined by a roller, a ball, or a band disposed about two rollers with parallel axes of rotation.

The container containing the cosmetic composition, in particular in the form of a fluid foundation, may optionally be integral with the applicator member during use or storage.

The applicator member is intended both to take a portion of the composition present in the associated container, and to apply the composition taken thereby to the surface of a keratinous material.

Firstly, the outer surface of the applicator member, and more particularly of the roller, is impregnated or coated with composition. The applicator member may be used to take the composition from a distinct container, or the composition may be delivered directly to the application surface by means of a container containing the composition to be applied and that is integral with said applicator member.

Secondly, the consecutive passage of the outer surface of the applicator member, more particularly of the roller, on the surface of a keratinous material, in particular the skin of the face, causes it to rotate and smoothes the composition disposed on its outer surface.

In variant exemplary embodiments, the applicator member may be provided with a removable brake that can block or brake free rotation thereof, if necessary, during removal and/or application.

Having regard to the various embodiments proposed below, in first variant exemplary embodiments these two steps, i.e. impregnation of the applicator member with the composition and application to the keratinous material, may be carried out consecutively. This happens when the container containing the composition is distinct from the applicator member. The user must initially bring the outer surface of the applicator member into contact with the composition to be taken. Depending on the texture of the composition, in particular its fluidity and viscosity, the applicator member may be impregnated and/or coated with the composition by rubbing and/or pressing the surface of the applicator member onto the surface of the composition, which may possibly cause it to rotate, or alternatively it may be “immersed” in the composition.

In other variant exemplary embodiments, impregnation of the applicator member with the composition and its movement over the keratinous material may be carried out simultaneously, as happens with applicator devices of the “distributor-applicator” tube type shown in FIG. 6, in which the composition to be applied is stored in a container that is integral with the applicator member and it is delivered directly to the surface of the applicator member.

The outer face of the applicator member is formed, at least on the surface, by a material that is capable of removing some cosmetic composition and of retaining it until it is subsequently applied to the skin.

For example, the application surface intended to come into contact with a keratinous material may in particular be a foam with optionally-flocked open or closed cells, a flocked material, an elastomer, a sintered material, a woven material or a nonwoven material.

The outer surface of the applicator member may optionally be smooth. Thus, said applicator member may advantageously have surface portions in relief, that are generally domed and rounded, which portions are advantageous in providing a simultaneous massaging effect.

In preferred variant exemplary embodiments, its appearance is close to that of an applicator roller similar to that used for applying paint. Such applicators for cosmetic purposes are described in particular in documents FR 985 064 (lipstick), FR 1 524 192 (powder), FR 1 281 338 (compact), FR 2 848 790 (solid or fluid cosmetic) and WO 2006/090061 (fluid).

Thus, the applicator member is advantageously in the form of a roller that may optionally be hollow and that rotates about an axis of rotation. Said axis of rotation may advantageously be disposed perpendicular to the longitudinal axis of the device.

In particular exemplary embodiments, the applicator device and the container are combined in the same packaging, for example the same case provided with a base containing the composition and having a recess for receiving the applicator and a cover that can move relative to the base, for example that is hinged thereto.

In a particularly preferred embodiment, the application surface of said applicator device is of the foam type, thereby different from plastic or metal materials generally used for rotating balls of roll-on dispensing systems, such as roll-on deodorants and antiperspirants.

In an other particularly preferred embodiment, the applicator means are advantageously in the form of a roller, thus different from a rotating ball.

In particular, the use of a roller presenting a larger application surface in contact with the keratinous material makes it possible to improve the quality of the final makeup, especially in terms of uniformity of the deposit.

During application, the applicator member is capable of turning on the skin without slipping. Several passes may be made in succession at the same location depending, for example, on the intensity of color desired. During successive passes, the user may modify the direction of rolling a little in order to blend out the edges of the deposited composition.

Such an applicator device is good to hold in the hand and very easy to use.

The mode of application of the invention may advantageously improve the sensory feelings provided by application of the emulsion to the keratinous material, in particular the skin, and more particularly may enhance the sensation of freshness or “fresh effect” felt by the user on application. In particular, the application of a composition of the invention with an applicator member in accordance with the invention, in particular of the foam type, enables to confer a sensation of freshness over a long time.

Further, using an applicator member of the invention to deposit a composition on the surface of a keratinous material, more particularly the skin, may produce a massaging effect on the skin that may produce sensations that are agreeable to the user.

The mode of application of the invention also has the advantage of not soiling the fingers with the composition on application.

Further, using an applicator member of the invention enables a makeup effect to be obtained that is significantly improved in terms of uniformity of the deposit, producing a discrete, natural makeup effect.

In other exemplary embodiments the invention provides a makeup and/or care method for a keratinous material, the method comprising the steps of:
taking a quantity of a composition, in particular in the form of fluid foundation, with the aid of an applicator member including an application surface that is capable of turning about an axis or a center of rotation, in particular as defined below, in response to being moved in engagement with the keratinous material; and
applying the composition as taken in this way to said keratinous material with the aid of said applicator member, said composition being in the form of a water-in-oil, oil-in-water or multiple emulsion comprising at least 30% by weight of aqueous phase relative to its total weight, and having viscosity measured at 25° C. and at a shear rate of 200 min⁻¹ of strictly more than 0.8 Pa·s.

### EXAMPLES OF APPLICATOR DEVICES AND OF COSMETIC KITS FOR CARRYING OUT THE INVENTION

The figures show some examples, inter alia, of applicator devices that may be used of the invention, and are particularly adapted to the application of a fluid composition with a greater or lesser viscosity.

In the drawings:

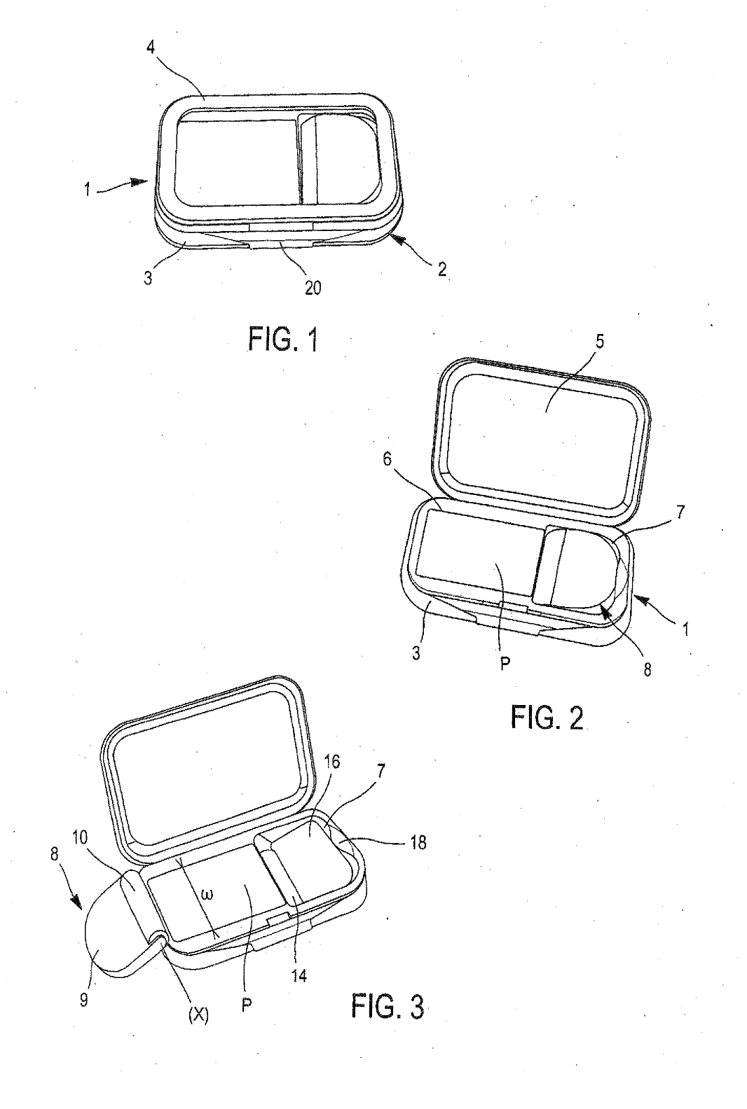

FIG. 1 is a diagrammatic perspective view of an example of a packaging and applicator device of the invention;

FIG. 2 shows the FIG. 1 device, with its case open;

FIG. 3 shows the FIG. 1 device, with its applicator device removed;

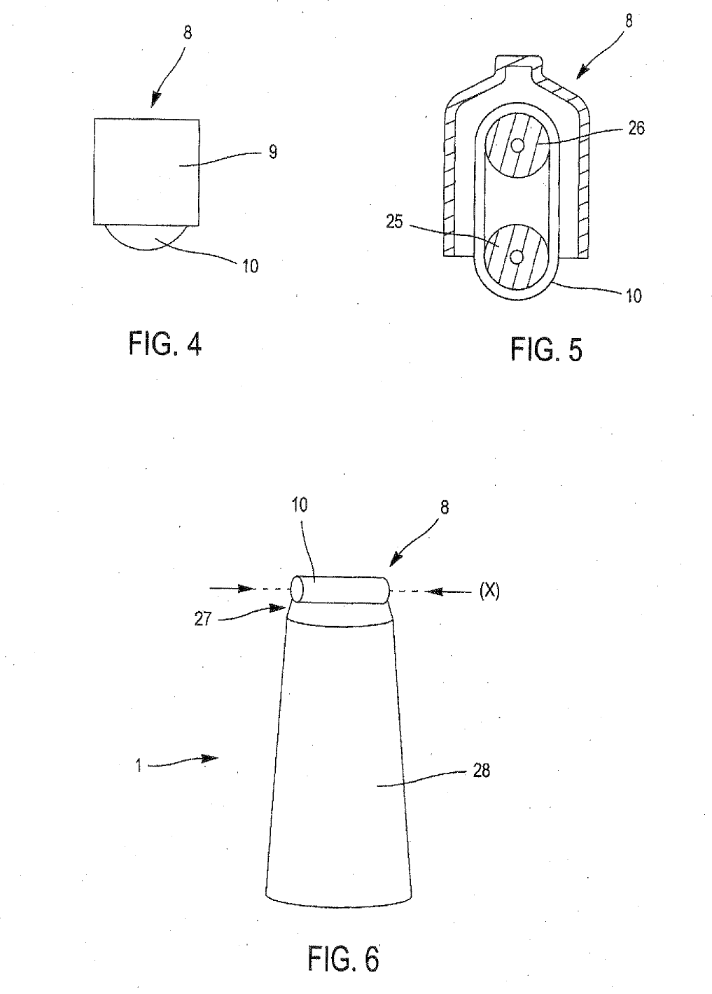

FIG. 4 is a diagrammatic elevation view of a variant embodiment of the applicator member;

FIG. 5 shows another variant embodiment of the applicator device, in diagrammatic, fragmentary longitudinal section;

FIG. 6 is a diagrammatic perspective view showing another example of a packaging and applicator device of the invention; and

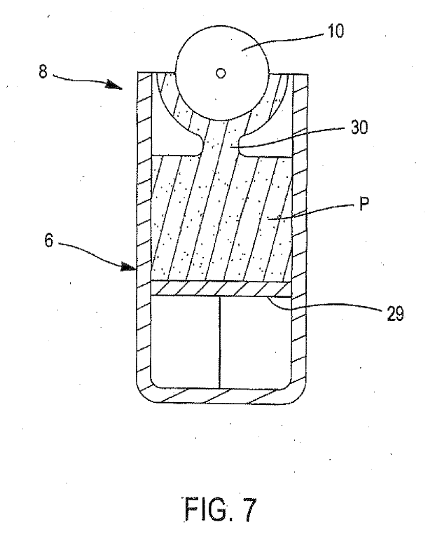

FIG. 7 shows another variant embodiment of the applicator device, in diagrammatic, fragmentary longitudinal section.

The packaging and applicator device 1 shown in FIGS. 1, 2, and/or 3 comprises a case 2 including a base and a lid 4 that is movable relative to the base; said lid 4 may comprise a transparent window 5 allowing the contents of the case to be seen when the case is closed.

The base 3 includes a recess 6 receiving the composition of the invention and a recess 7 accommodating an applicator device 8, acting to take the composition P and to apply it to human keratinous material.

The applicator device 8 comprises a handle 9 and an applicator member 10 capable of turning relative to the handle 9, for example and as shown, about an axis of rotation X that may be oriented perpendicularly to the longitudinal axis of the handle. This handle may have a shape that is generally flat parallel to a plane containing the axis of rotation X.

The applicator member 10 may be in the form of a roller with an application surface that may be a cylinder of revolution about the axis X.

The application surface may be defined by any material capable of retaining the composition and of applying it, for example an elastomeric material or a foam with open or closed cells, or a flocked membrane. The material defining the application surface may be compressible.

The length of the applicator member 10 may correspond substantially to the width w of the recess 6 containing the composition.

The recess 7 receiving the applicator device 8 may have a bottom 14 provided with a grid 14 and may include a slope 16 that keeps the handle 9 inclined slightly upwardly and towards the side of the case, to facilitate grasping by the user. A cutout 18 may open into the recess 7 in order to facilitate engagement of a finger in order to pick up the handle 9.

The recess 6 receiving the composition P may be parallelepipedal in shape or have a concave rounded shape that is a portion of a cylinder with a generator line parallel to the axis of rotation X when the applicator device 8 is moved in the recess 6 containing the composition in order to load it with composition. This avoids the presence of corners where the composition could accumulate without being taken.

The case may include a clasp 20 that may be of any type.

To use the applicator device 8, the user brings the applicator member 10 into contact with the composition P contained in the container 6, by moving it to a greater or lesser depth in the container 6, or at the surface of the composition P, and then applies it to the keratinous material to be treated, for example the skin, by passing it one or more times over the zone where the composition is to be deposited.

During movement over the skin, the applicator member 10 can roll without slipping.

The invention is not limited to an applicator member in the form of a roller turning about an axis of rotation.

As an example, as shown in FIG. 4, the applicator member 10 may be in the form of an applicator ball 10, which may be retained in a cage, not shown, that is fastened to a handle 9. In the example of FIG. 4, the applicator member 10 is capable of turning about any axis in the cage.

In a variant embodiment shown in FIG. 5, the applicator member 10 can also be defined by an applicator band mounted to turn about two rollers 25 and 26 with parallel axes of rotation. The portion of the band 10 acting in application may be moved in contact with the skin when the applicator device 10 is moved in contact therewith to apply the composition.

In a variant embodiment, not shown, the axes of rotation of the rollers 25 and 26 are disposed in such a manner that the portion of the band 10 that comes into contact with the keratinous material in order to apply the composition is not in contact with only one roller but is between both rollers.

In a variant embodiment, the packaging and applicator device 1 shown in FIG. 6 is in the form of a distributor-applicator tube adapted to store, dispense and apply a fluid composition P of greater or lesser viscosity.

Such applicator-distributor tubes are described in particular in patent application WO 2006/090061.

Such a device may comprise a container for storing the composition, the container in the form of a flexible tube 28 including, at its end, a tip 27 with an applicator device 8.

The flexible tube 28 may take any form and be made of various materials, especially plastics or thermoplastic materials.

Advantageously, the tip is surmounted by a suitable cap, not shown, that protects the applicator device during storage of the tube.

The applicator device 8 of the invention includes an applicator member 10 that may be in of form of a roller having an application surface that may be a cylinder of revolution about the axis X, as shown in FIG. 6. This applicator member is capable of turning and of taking the composition from the container on one side while applying it on the other side.

In a variant embodiment, not shown, the applicator member of the tip of the dispenser-applicator tube may be an applicator ball as can be seen in FIG. 4.

In one possible variant embodiment of the packaging and applicator device shown in FIG. 6, the tip 27 may include a hollow compartment, not shown, connected with the interior of said tube 28. The applicator roller 10 can thus turn in said hollow compartment, be supplied with composition, and transport the composition to the keratinous material.

To use the packaging and applicator device shown in FIG. 6, and in order to load the tip 27 with composition, the user presses on the flexible wall of the tube 28 while continuing to apply said applicator member 8 to the keratinous material to be treated, for example the skin.

In yet another variant embodiment, the packaging and applicator kit shown in FIG. 7 may include a container 6 for storing the composition P, and in which a piston 29 may slide to force the composition P towards the applicator member 10 that is fed via at least one internal channel 30. The applicator member 10 may be as defined above, for example a ball.

In variant embodiments that are not shown, the applicator device 8 may be provided with a brake to brake or even prevent rotation of the applicator member 10 relative to the handle.

Composition

Aqueous Phase

As discussed above, the aqueous phase forms at least 30% by weight of the total weight of the composition of the invention.

In a particular embodiment, the aqueous phase constitutes in the range 30% to 70% by weight, more particularly at least 32% by weight, in particular in the range 35% to 45% by weight relative to the total weight of the composition of the invention.

The aqueous phase comprises water and/or at least one hydrosoluble solvent.

The water may be a floral water such as cornflower water and/or a mineral water such as VITTEL water, LUCAS water or LA ROCHE POSAY water, and/or spa water.

The term “hydrosoluble solvent” as used in the present invention means a compound that is liquid at ambient temperature and miscible with water (miscibility in water of more than 50% by weight at 25° C. and at atmospheric pressure).

In particularly preferred exemplary embodiments, the aqueous phase of a composition of the invention comprises at least one C₂ to C₈ lower mono-alcohol.

More particular lower mono-alcohols that are suitable for the invention comprise in the range 2 to 8 carbon atoms, in particular in the range 2 to 6 carbon atoms, and more particularly, in the range 2 to 4 carbon atoms, such as ethanol, isopropanol, propanol or butanol.

Ethanol and isopropanol, and preferably ethanol, are particularly suitable for use in the invention.

The emulsion of the invention may comprise one or more lower C₂ to C₈ mono-alcohols in a quantity of at least 2%, in particular at least 3%, more particularly in the range 5% to 7% by weight relative to the total weight of the composition.

In addition to the lower monoalcohol(s) defined above, this aqueous phase may contain other alcohol(s), in particular polyethylene glycols containing 6 to 80 ethylene oxide units; polyols such as propylene glycol, isoprene glycol, butylene glycol, glycerin, sorbitol, glycols such as propylene glycol, butylene glycol, dipropylene glycol, diethylene glycol, glycol ethers such as the (C₁-C₄) alkyl ethers of mono-, di- or tri-propylene glycol or mono-, di- or tri-ethylene glycol, and mixtures thereof.

The aqueous phase may further include stabilizing agents, for example sodium chloride, magnesium dichloride and magnesium sulfate.

The aqueous phase may also include any hydrosoluble or hydrodispersible compound that is compatible with an aqueous phase, such as gelling agents, film-forming polymers, thickening agents, surfactants and mixtures thereof.

Fatty Phase

The fatty phase may represent in the range 20% to 40%, preferably in the range 22% to 30% by weight, more preferably in the range 22% to 26% by weight relative to the total weight of the emulsion.

A composition of the invention may comprise at least one oil, selected from volatile and non-volatile oils of the hydrocarbon, silicone or fluorinated type. The oils may be of animal, vegetable, mineral or synthetic origin.

The term “oil” means any fat in the liquid form at ambient temperature (20° C.-25° C.) and at atmospheric pressure.

The term “volatile oil” in the context of the invention means an oil that evaporates in contact with keratinous material in less than one hour, at ambient temperature and atmospheric pressure. The volatile oil is a volatile cosmetic oil that is liquid at ambient temperature, having non-zero vapor pressure at ambient temperature and atmospheric pressure, in particular in the range 0.13 Pa [pascal] to 40,000 Pa (10⁻³ mmHg [millimeter of mercury] to 300 mmHg), preferably having a vapor pressure in the range 1.3 Pa to 13,000 Pa (0.01 mmHg to 100 mmHg), and more preferably in the range 1.3 Pa to 1300 Pa (0.01 mmHg to 10 mmHg).

Further, the volatile oil generally has a boiling point, measured at atmospheric pressure, in the range 150° C. to 260° C., preferably in the range 170° C. to 250° C.

Advantageously, the fatty phase comprises in the range 15% to 40% by weight, preferably in the range 20% to 35% by weight, and more preferably in the range 17% to 30% by weight of volatile oil(s) relative to the total weight of the composition.

The term “hydrocarbon oil” means an oil essentially formed or even constituted by carbon atoms and hydrogen, and possibly oxygen atoms, nitrogen atoms, and containing no silicon or fluorine; it may contain ester, ether, amine or amide groups.

The term “silicone oil” means an oil containing at least one silicon atom, and especially containing Si—O groups.

The term “fluorinated oil” means an oil containing at least one fluorine atom.

The volatile hydrocarbon oil for use in the invention may be selected from hydrocarbon oils with a flash point in the range 40° C. to 102° C., preferably in the range 40° C. to 55° C., and more preferably in the range 40° C. to 50° C.

Examples of volatile hydrocarbon oils that may be mentioned are volatile hydrocarbon oils containing 8 to 16 carbon atoms and mixtures thereof, and in particular branched C₈-C₁₆ alkanes such as C₈-C₁₆ isoalkanes (also termed isoparaffins), isododecane, isodecane, isohexadecane and, for example, oils sold under the commercial names Isopars or Permetyls, branched C₈-C₁₆ esters such as iso-hexyl neopentanoate, and mixtures thereof. Preferably, the hydrocarbon volatile oil is selected from volatile hydrocarbon oils containing 8 to 16 carbon atoms and mixtures thereof, in particular isododecane, isodecane, isohexadecane, in particular isododecane.

The volatile hydrocarbon oil may be present in a quantity in the range 5% to 20% by weight relative to the total weight of the emulsion, preferably in the range 6% to 15% by weight, and more preferably in the range 7% to 10% by weight.

The volatile silicone oil for use in the invention may be selected from silicone oils with a flash point in the range 40° C. to 102° C., preferably with a flash point of more than 55° C. and 95° C. or less, and more preferably in the range 65° C. to 95° C.

Examples of silicon oils that may be mentioned are linear or cyclic silicone oils containing 2 to 7 silicon atoms, these silicones optionally comprising alkyl or alkoxy groups containing 1 to 10 carbon atoms. Examples of volatile silicone oils that may be mentioned in particular are octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

Examples of volatile fluorinated oils that may be mentioned are nonafluoroethoxybutane, nonafluoromethoxybutane, decafluoropentane, tetradecafluorohexane, dodecafluoropentane and mixtures thereof.

The volatile oil selected from volatile silicone oils, volatile fluorinated oils and mixtures thereof may be present in a quantity in the range 10% to 32% by weight relative to the total weight of the emulsion, preferably in the range 15% to 30% by weight, and more preferably in the range 17% to 26% by weight relative to the total weight of the composition.

The fatty phase of the emulsion of the invention may further comprise at least one non-volatile oil.

This non-volatile oil or one of its mixtures may be present in a quantity in the range 0.1% to 40% by weight relative to the total weight of the emulsion, preferably in the range 1% to 25% by weight.

The non-volatile oil may be selected from oils of mineral, animal, vegetable or synthetic origin, from carbonaceous, hydrocarbon and/or silicone oils, and mixtures thereof, provided that they are compatible with the envisaged use.

Examples of non-volatile hydrocarbon oils that may be mentioned are paraffin or Vaseline oil, isoeicosane, mink, tortoise, soy, perhydrosqualene and sweet almond oils, calophyllum, palm, grapeseed, sesame, corn, arara, rapeseed, sunflower, cotton, apricot, castor, avocado, jojoba, olive or cereal germ oils; esters of lanolic acid, oleic acid, lauric acid, stearic acid; fatty esters such as isopropyl myristate, isopropyl palmitate, butyl stearate, hexyl laurate, diisopropyl adipate, isononyl isononanoate, 2-ethyl-hexyl palmitate, 2-hexyl-decyl laurate, 2-octyl-decyl palmitate, 2-octyl-dodecyl myristate or lactate, 2-diethyl-hexyl succinate, diisostearyl malate, glycerin or diglycerin triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl or linolenyl alcohol isostearyl alcohol or octyl dodecanol.

Non-volatile silicone oils that may be mentioned are polydimethylsiloxane (PDMS), optionally phenylated, such as phenyltrimethicones, or optionally substituted with aliphatic and/or aromatic groups or with functional groups such as hydroxyl, thiol and/or amine groups; polysiloxanes modified with fatty acids, fatty alcohols or polyoxyalkylenes, and mixtures thereof.

The fatty phase may also comprise at least one wax, at least one gum and/or at least one pasty fat of vegetable, animal, mineral or synthetic origin, or even siliconized, and mixtures thereof.

Examples of waxes that are solid at ambient temperature that may be present in the composition of the invention are that may be mentioned are hydrocarbon waxes such as beeswax, Carnauba wax, candellila, ouricoury, Japan wax, cork or sugar cane fiber waxes, paraffin waxes, lignite waxes, microcrystalline waxes, lanolin wax, Montan wax, ozokerites, polyethylene waxes, waxes obtained by Fischer-Tropsch synthesis, hydrogenated oils and fatty esters and glycerides that are solid at 25° C. It is also possible to use silicone waxes; examples that may be mentioned are alkyl or alkoxy polymethylsiloxanes and/or polymethylsiloxane esters. The waxes may be in the form of stable dispersions of colloidal particles of wax, such as those that can be prepared using known methods such as those in “Microemulsions Theory and Practice”, L. M. Prince Ed., Academic Press (1977), pp 21-32. A wax that is liquid at ambient temperature and that may be mentioned is Jojoba oil.

The waxes may be present in the range 0.1% to 10% by weight relative to the total weight of the emulsion, and preferably in the range 0.1% to 5% by weight.

Pasty fatty compounds can be defined with the aid of at least one of the following physico-chemical properties:
a viscosity in the range 0.1 Pa·s to 40 Pa·s (1 poise to 400 poises), preferably 0.5 Pa·s to 25 Pa·s, measured at 40° C. with a CONTRAVES TV rotary viscosimeter equipped with a MS-r3 or MS-r4 moving body at a frequency of 60 Hz [hertz];
a melting point in the range 25° C. to 70° C., preferably 25° C. to 55° C.

The compositions of the invention may also comprise at least one alkyl-, alkoxy- or phenyl-dimethicone such as, for example, the composition sold under the name “Abil wax 2440” by the supplier GOLDSCHMIDT.

The emulsion of the invention may also comprise a thickening agent for the fatty phase. The thickening agent may be selected from:
organomodified clays; these are clays treated with compounds primarily selected from quaternary amines and tertiary amines. Examples of organomodified clays that may be mentioned are organomodified bentonites such as those sold under the name “Bentone 34” by the supplier RHEOX, and organomodified hectorites such as those sold under the name “Bentone 27”, “Bentone 38” by the supplier RHEOX;
hydrophobic pyrogenated silica. Such silicas are, for example sold in particular under the references “AEROSIL R812®” by the supplier Degussa, “CAB-O-SIL TS-530®” by the supplier Cabot and under the references “AEROSIL R972®”, “AEROSIL R974®” by the supplier Degussa, “CAB-O-SIL TS-610®”, “CAB-O-SIL TS-720®” by the supplier Cabot.

The thickening agent for the fatty phase may be present in a quantity in the range 0.1% to 5% by weight relative to the total weight of the emulsion, and more preferably from 0.4% to 3% by weight.

Surfactants

A composition of the invention may comprise one or more surfactant(s), in particular selected from amphoteric, anionic, cationic or non-ionic surfactants, used alone or as a mixture.

The surfactants are generally present in the composition in a quantity that may be in the range 3% to 20% by weight, in particular in the range 3.5% to 15% by weight, or even strictly more than 5% by weight, and more particularly in the range 5.1% to 10% by weight relative to the total weight of the composition.

In particular exemplary embodiments, in particular when the composition of the invention is a W/O emulsion, the composition may include at least one silicone surfactant.

The silicone surfactant or surfactants may be present in a quantity in the range 0.5% to 10% by weight, in particular in the range 1% to 6% by weight relative to the total weight of the composition.

In particular exemplary embodiments, the non-silicone surfactant(s) may be present in a quantity in the range 4% to 10% by weight, in particular from 5% to 8% by weight relative to the total weight of the composition.

Clearly, the surfactant is selected in a manner that can effectively stabilize the emulsions under particular consideration in the context of the invention, namely of the O/W, W/O, or O/W/O type. This choice falls within the competence of the skilled person.

As regards O/W emulsions, mention may be made, for example, of non-ionic surfactants, and in particular esters of polyols and fatty acids with a saturated or unsaturated chain containing, for example, 8 to 24 carbon atoms and more preferably 12 to 22 carbon atoms, and oxyalkylenated derivatives thereof, i.e. comprising oxyethylenated and/or oxypropylenated units, such as esters of glycerol and C₈-C₂₄ fatty acids, and oxyalkylenated derivatives thereof; esters of polyethylene glycol and C₈-C₂₄ fatty acids, and oxyalkylenated derivatives thereof; esters of sorbitol and C₈-C₂₄ fatty acids, and oxyalkylenated derivatives thereof; esters of sugar (sucrose, glucose, alkylglucose) and C₈-C₂₄ fatty acids, and oxyalkylenated derivatives thereof; oxyethylenated ethers of fatty acids and sugars (glucose, alkylglucose); sucrose esters; fatty alcohol ethers; ethers of sugar and of C₈-C₂₄ fatty alcohols, and mixtures thereof.

Examples of esters of glycerol and fatty acid that may be mentioned in particular are glyceryl stearate (glyceryl mono-, di- and/or tri-stearate) (CTFA name: glyceryl stearate) or glyceryl ricinoleate, and mixtures thereof.

Examples of esters of polyethylene glycol and fatty acid that may be mentioned in particular are polyethylene glycol stearate (polyethylene glycol mono-, di- and/or tri-stearate), and more particularly polyethylene glycol 50 OE monostearate (CTFA name: PEG-50 stearate), polyethylene glycol 100 OE monostearate (CTFA name: PEG-100 stearate) and mixtures thereof.

It is also possible to use mixtures of said surfactants such as the product containing glyceryl stearate and PEG-100 stearate sold under the name ARLACEL 165 by the supplier Uniqema, and the composition containing glyceryl stearate (glyceryl mono-distearate) and potassium stearate, sold under the name TEGIN by the supplier Goldschmidt (CTFA name: glyceryl stearate SE).

Examples of esters of fatty acid and glucose or alkylglucose that may be mentioned in particular are glucose palmitate, alkylglucose sesquistearates such as methylglucose sesquistearate, alkylglucose palmitates such as methylglucose or ethylglucose palmitate, fatty esters of methylglucoside and more particularly the diester of methylglucoside and of oleic acid (CTFA name: Methyl glucose dioleate); the mixed ester of methylglucoside and an oleic acid/hydroxystearic acid mixture (CTFA name: Methyl glucose dioleate/hydroxystearate); the ester of methylglucoside and of isostearic acid (CTFA name: Methyl glucose isostearate); the ester of methylglucoside and of lauric acid (CTFA name: Methyl glucose laurate); the mixture of the monoester and diester of methylglucoside and isostearic acid (CTFA name: Methyl glucose sesqui-isostearate); the mixture of the monoester and diester of methylglucoside and stearic acid (CTFA name: Methyl glucose sesquistearate) and in particular the product sold under the name Glucate SS by the supplier AMERCHOL, and mixtures thereof.

Examples of oxyethylenated ethers of fatty acid and glucose or alkylglucose that may be mentioned are oxyethylenated ethers of fatty acids and methylglucose, and in particular the polyethylene glycol ether of the diester of methyl glucose and stearic acid with approximately 20 moles of ethylene oxide (CTFA name: PEG-20 methyl glucose distearate), such as the composition sold under the name Glucam E-20 distearate by the supplier AMERCHOL; the polyethylene glycol ether of the mixture of the monoester and diester of methyl glucose and stearic acid containing approximately 20 moles of ethylene oxide (CTFA name: PEG-20 methyl glucose sesquistearate) and in particular the product sold under the name Glucamate SSE-20 by the supplier AMERCHOL and such as that sold under the name Grillocose PSE-20 by the supplier GOLDSCHMIDT, and mixtures thereof.

Examples of sucrose esters that may be mentioned are saccharose palmito-stearate, saccharose stearate and saccharose monolaurate.

Examples of ethers of fatty alcohols that may be mentioned are the ethers of polyethylene glycol and fatty alcohol containing 8 to 30 carbon atoms, and in particular 10 to 22 carbon atoms, such as the ethers of polyethylene glycol and cetyl, stearyl, cetearyl alcohols (mixture of cetyl and stearyl alcohols). Mention may be made, for example, of ethers containing 1 to 200 and preferably 2 to 100 oxyethylenated groups, such as those with CTFA names Ceteareth-20, Ceteareth-30, and mixtures thereof.

Examples of ethers of sugars that may be mentioned in particular are alkylpolyglucosides, for example decylglucoside, such as the product sold under the name MYDOL 10 by the supplier Kao Chemicals, the product sold under the name PLANTAREN 2000 by the supplier Henkel, and the product sold under the name ORAMIX NS 10 by the supplier Seppic; caprylyl/capryl glucoside, such as the product sold under the name ORAMIX CG 110 by the supplier Seppic or under the name LUTENSOL GD 70 by the supplier BASF; laurylglucoside, such as the products sold under the names PLANTAREN 1200 N and PLANTACARE 1200 by the supplier Henkel; coco-glucoside, such as the product sold under the name PLANTACARE 818/UP by the supplier Henkel; cetostearyl glucoside, optionally as a mixture with cetostearyl alcohol, sold for example under the name MONTANOV 68 by the supplier Seppic, under the name TEGO-CARE CG90 by the supplier Goldschmidt and under the name EMULGADE KE3302 by the supplier Henkel; arachidyl glucoside, for example in the form of a mixture of arachidic and behenic alcohols and arachidyl glucoside sold under the name MONTANOV 202 by the supplier Seppic; cocoylethylglucoside, for example in the form of a (35/65) mixture with cetyl and stearyl alcohols, sold under the name MONTANOV 82 by the supplier Seppic, and mixtures thereof.

Particular W/O emulsions that may be used are hydrocarbon or silicon surfactants.

Examples of hydrocarbon surfactants that may be mentioned are polyesters of polyols such as PEG-30 dipolyhydroxystearate sold under the reference ARLACEL P 135 by the supplier Uniqema, polyglyceryl-2 dipolyhydroxystearate sold under the reference DEHYMULS PGPH by the supplier Cognis.

Examples of silicone surfactants that may be mentioned are alkyl-dimethicone copolyols such as laurylmethicone copolyol sold under the name “Dow Corning 5200 Formulation Aid” by the supplier Dow Corning and cetyl dimethicone copolyol sold under the name ABIL EM 90 by the supplier Goldschmidt, or the polyglyceryl-4 isostearate/cetyl dimethicone copolyol/hexyl laurate mixture sold under the name ABIL WE 09 by the supplier Goldschmidt.

One or more co-emulsifying agents may also be added. Advantageously, the co-emulsifying agent may be selected from the group comprising calculated esters of polyol. Examples of alkylated esters of polyol that may be mentioned are esters of glycerol and/or Sorbian, for example polyglyceryl-3 diisostearate sold in particular under the name LAMEFORM TGI by the supplier Cognis, polyglycerol-4 isostearate, such as the product sold under the name Isolan GI 34 by the supplier Goldschmidt, sorbitan isostearate, such as the product sold under the name Arlacel 987 by the supplier ICI, and sorbitan and glycerol isostearate, such as the product sold under the name Arlacel 986 by the supplier ICI, and mixtures thereof.

As surfactants for W/O emulsions, it is also possible to use a solid cross-linked organopolysiloxane elastomer comprising at least one oxyalkylene group, such as those obtained using the operating procedure of Examples 3, 4 and 8 of document U.S. Pat. No. 5,412,004 and the examples in the document U.S. Pat. No. 5,811,487, in particular the product of Example 3 (synthesis example) in patent U.S. Pat. No. 5,412,004 and that sold in particular under the reference KSG 21 by the supplier Shin Etsu.

In particularly preferred exemplary embodiments, an emulsion of the invention, in particular a W/O emulsion, includes at least one silicone surfactant, and more particularly selected from dimethicone copolyols.

The presence of a dimethicone copolyol in particular encourages stabilization of the emulsion of the invention.

A dimethicone copolyol that can be used in the invention is an oxypropylenated and/or oxyethylenated polydimethyl methyl siloxane.

The dimethicone copolyol used may have the following formula (II):

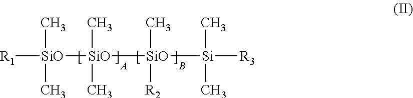

in which:
R₁, R₂, R₃, independently of one another, represent a C₁-C₆ alkyl radical or a —(CH₂)ₓ—(OCH₂CH₂)_{y}—(OCH₂CH₂CH₂)_{z}—OR₄ radical, at least one of the radicals R₁, R₂ or R₃ not being an alkyl radical; R₄ being a hydrogen, a C₁-C₃ alkyl radical or a C₂-C₄ acyl radical;
A is an integer in the range 0 to 200;
B is an integer in the range 0 to 50; provided that A and B are not zero at the same time;
x is an integer in the range 1 to 6;
y is an integer in the range 1 to 30; and z is an integer in the range 0 to 5.

In preferred exemplary embodiments of the invention, in the compound with formula (II), R₁═R₃=methyl radical, x is an integer in the range 2 to 6 and y is an integer in the range 4 to 30. In particular, R₄ is a hydrogen.

Examples of compounds with formula (II) that may be mentioned are compounds with formula (III):

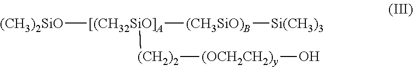

in which A is an integer in the range 20 to 105, B is an integer in the range 2 to 10 and y is an integer in the range 10 to 20.

Examples of silicon compounds with formula (II) that may be mentioned are compounds with formula (IV):

HO—(OCH₂CH₂)_{y}—(CH₂)₃—[(CH₃)₂SiO]_{A′}—(CH₂)₃—(OCH₂CH₂)_{y}—OH (IV)

in which A′ and y are integers in the range 10 to 20.

Examples of dimethicone copolyols that may be mentioned are those sold under the names DC 5329, DC 7439-146, DC 2-5695, Q4-3667 by the supplier Dow Corning; KF-6013, KF-6015, KF-6016, KF-6017, KF-6028 by the supplier Shin-Etsu.

The compounds DC 5329, DC 7439-146, DC 2-5695 are compounds with formula (III) in which respectively A is 22, B is 2 and y is 12; A is 103, B is 10 and y is 12; A is 27, B is 3 and y is 12.

In particular exemplary embodiments, the silicone surfactant may be PEG-polydimethyl/siloxyethyl/dimethicone, sold in particular by the supplier Shin-Etsu under the reference KF-6028, PEG-10 dimethicone sold in particular by the supplier Shin-Etsu under the reference KF-6017, and mixtures thereof.

The dimethicone copolyol may be present in the composition of the invention in a quantity in the range 1% to 6% by weight relative to the total weight of the emulsion, and preferably in the range 1.5% to 4% by weight, and more preferably in the range 2% to 3% by weight.

In preferred variant exemplary embodiments of the invention, the above-mentioned dimethicone copolyol may be associated with at least one α-ω substituted oxyalkylenated silicone.

α-ω Substituted Oxyalkylenated Silicone

Throughout this text, as is the usual practice, the term “silicone” designates any organosilicon polymer or oligomer with a linear or cyclic structure, branched or cross-linked, with a variable molecular weight, obtained by polymerization and/or polycondensation of silanes that are suitably functionalized, and essentially constituted by repeated principal motifs in which the silicon atoms are bonded together via oxygen atoms (siloxane bond, ≡Si—O—Si≡), the optionally substituted hydrocarbon radicals being directly bonded via a carbon atom to said silicon atoms. The most routine hydrocarbon radicals are alkyl radicals, in particular C₁-C₁₀, in particular methyl, fluoroalkyl radicals, and aryl radicals, in particular phenyl radicals. They may, for example, be substituted with C₁-C₄₀ ester or ether groups or C₇-C₆₀ aralkyl groups.

Thus, the α-ω substituted oxyalkylenated silicone of the invention is an organosilicon polymer as defined above, with a linear structure, substituted at both ends of the principal chain with oxyalkylene groups bonded to the Si atoms via a hydrocarbon group.

Preferably, the α-ω substituted oxyalkylenated silicone has the following general formula (I):

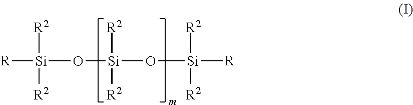

in which: R═—(CH₂)ₚO—(C₂H₄O)ₓ(C₃H₆O)_{y}R¹

where:
R¹ represents H, CH₃ or CH₂CH₃;
p is an integer in the range 1 to 5, x lies in the range 1 to 100, y lies in the range 0 to 50;
the units (C₂H₄O) and (C₃H₆O) may be distributed randomly or in blocks;
radicals R² represent a C₁-C₃ alkyl radical or a phenyl radical; and
5≦m≦300.

Preferably, the α-ω substituted oxyalkylenated silicone of the present invention has general formula (I) in which all of the radicals R² are methyl radicals; and
p is in the range 2 to 4;
x is in the range 3 to 100;
m is in the range 50 to 200.

More preferably, the mean molecular weight of R is in the range 800 to 2600.

Preferably, the weight ratio of the C₂H₄O units relative to the C₃H₆O units is in the range 100:10 to 20:80. Advantageously, this ratio is approximately 42/58.

More preferably, R¹ is the methyl group.

Still more preferably, the emulsion of the invention comprises the α-ω substituted oxyalkylenated silicone with the following formula:

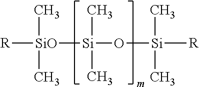

in which:
m=100;
R=(CH₂)₃—O—(C₂H₄O)ₓ—(C₃H₆O)_{y}—CH₃, where x is in the range 3 to 100, y is in the range 1 to 50, the weight ratio of the number of C₂H₄O to the number of C₃H₆O being approximately 42/58, the mean molecular weight of R being in the range 800 to 1000.

The α-ω substituted oxyalkylenated silicone as defined above may be used of the invention in a proportion in the range 0.5% to 5%, in particular from 1% to 4% by weight and more particularly from 2% to 3% by weight relative to the total weight of the composition.

Examples of commercially available products that may completely or partially contain α-ω substituted oxyalkylenated silicones that may be used of the invention as an emulsifying agent that may be mentioned are those sold under the names “Abil EM 97” by the supplier Goldschmidt, or “KF 6009”, “X22-4350”, “X22-4349” or “KF 6008” by the supplier Shin Etsu.

In particular, it may be cetyl dimethicone copolyol.

Emulsifying Silicone Elastomers

In other exemplary embodiments of the invention, the dimethicone copolyol that may be used in the invention may be associated with at least one emulsifying silicone elastomer.

The emulsifying silicon elastomer may be selected from a polyoxyalkylenated silicone elastomer, a polyglycerolated silicone elastomer and mixtures thereof.

Preferably, a polyglycerolated silicone elastomer is used.

The polyoxyalkylenated silicone elastomer is a cross-linked organopolysiloxane that may be obtained by addition cross-linking of diorganopolysiloxane containing at least one hydrogen bonded to silicon and a polyoxyalkylene having at least two groups with an ethylene type unsaturated bond.

Preferably, the polyoxyakylenated cross-linked organopolysiloxane is obtained by cross-linking addition reaction (A1) of a diorganopolysiloxane containing at least two hydrogens each bonded to a silicon, and (B1) with polyoxyakylene having at least two groups with an ethylene type unsaturated bond, in particular in the presence (C1) of a platinum catalyst as described, for example, in patents U.S. Pat. No. 5,236,986 and U.S. Pat. No. 5,412,004.

In particular, the organopolysiloxane may be obtained by reacting polyoxyakylene (in particular polyoxyethylene and/or polyoxypropylene) with dimethylvinylsiloxy ends and methylhydrogenopolysiloxane with trimethylsiloxy ends in the presence of a platinum catalyst.

The organic groups bonded to the silicon atoms of compound (A1) may be alkyl groups containing 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl, or 3,3,3-trifluoropropyl; aryl groups such as phenyl, tolyl or xylyl; substituted aryl groups such as phenylethyl; and monovalent substituted hydrocarbon groups such as an epoxy group, an ester carboxylate group or a mercapto group.

The compound (A1) may thus be selected from methylhydrogenopolysiloxanes with trimethylsiloxy ends, dimethylsiloxane-methylhydrogenosiloxane copolymers with trimethylsiloxy ends, cyclic dimethylsiloxane-methylhydrogenosiloxane copolymers, and dimethylsiloxane-methylhydrogenosiloxane-laurylmethylsiloxane copolymers with trimethylsiloxy ends.

The compound (C1) is the catalyst for the cross-linking reaction and is in particular chloroplatinic acid, chloroplatinic acid-olefin complexes, chloroplatinic acid-alkenylsiloxane complexes, chloroplatinic acid-diketone complexes, platinum black, or supported platinum.

Advantageously, the polyoxyalkylenated silicone elastomers are formed from divinyl compounds, in particular polyoxyalkylenes with at least two vinyl groups, reacting with the Si—H bonds of a polysiloxane.

The polyoxyalkylenated silicone elastomer of the invention is preferably mixed with at least one hydrocarbon oil and/or a silicone oil to form a gel. In said gels, the polyoxyalkylenated elastomer is in the form of non-spherical particles.

Polyoxyalkylenated silicone elastomers are described in particular in patents U.S. Pat. No. 5,236,986, U.S. Pat. No. 5,412,004, U.S. Pat. No. 5,837,793, and U.S. Pat. No. 5,811,487, of contents that are hereby incorporated by reference.

Examples of polyoxyalkylenated elastomer silicones that may be used are those sold in particular under the names “KSG-21”, “KSG-20”, “KSG-30”, “KSG-31”, “KSG-32”, “KSG-33”, “KSG-210”, “KSG-310”, “KSG-320”, “KSG-330”, “KSG-340”, “X-226146” by the supplier Shin Etsu, “DC9010”, “DC9011” by the supplier Dow Corning.

In other exemplary embodiments, the emulsifying silicone elastomer may advantageously be selected from polyglycerolated silicone elastomers.

The polyglycerolated silicone elastomer is a cross-linked organopolysiloxane elastomer that may be obtained by addition cross-linking of a diorganopolysiloxane containing at least one hydrogen bonded to silicon and polyglycerolated compounds containing groups with an ethylenically unsaturated bond, in particular in the presence of a platinum catalyst.

Preferably, the cross-linked organopolysiloxane elastomer is obtained by a cross-linking addition reaction (A) of diorganopolysiloxane containing at least two hydrogens each bonded to a silicon, and (B) glycerolated compounds containing at least two groups with an ethylene type unsaturated bond, in particular in the presence (C) of a platinum catalyst.

In particular, the organopolysiloxane may be obtained by reaction of a polyglycerolated compound with dimethylvinylsiloxy ends and methylhydrogenopolysiloxane with trimethylsiloxy ends, in the presence of a platinum catalyst.

The compound (A) is the base reagent for the formation of the organopolysiloxane elastomer and cross-linking is carried out by an addition reaction of compound (A) with compound (B) in the presence of catalyst (C).

In particular, compound (A) is an organopolysiloxane containing at least two hydrogen atoms bonded to distinct silicon atoms in each molecule.

In particular, compound (A) may have any molecular structure, especially a linear or branched chain structure or a cyclic structure.

Compound (A) may have a viscosity at 25° C. in the range 1 cSt [centistoke] to 50,000 cSt, in particular, so that it is properly miscible with compound (B).

The organic groups bonded to the silicon atoms of compound (A) may be alkyl groups containing 1 to 18 carbon atoms, such as methyl, ethyl, propyl, butyl, octyl, decyl, dodecyl (or lauryl), myristyl, cetyl, stearyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, a carboxylate ester group, or a mercapto group. Preferably, said organic group is selected from methyl, phenyl and lauryl groups.

Compound (A) may thus be selected from methylhydrogenopolysiloxanes with trimethylsiloxy ends, dimethylsiloxane-methylhydrogenosiloxane copolymers with trimethylsiloxy ends, cyclic dimethylsiloxane-methylhydrogenosiloxane copolymers, and dimethylsiloxane-methylhydrogenosiloxane-laurylmethylsiloxane copolymers with trimethylsiloxy ends.

Compound (B) may be a polyglycerolated compound with formula (B′) below:

CₘH₂ₘ₋₁—O-[Gly]ₙ-CₘH₂ₘ₋₁ (B′)

in which m is an integer in the range 2 to 6, n is an integer in the range 2 to 200, preferably in the range 2 to 100, more preferably in the range 2 to 50, preferably with n in the range 2 to 20, preferably in the range 2 to 10, and more preferably in the range 2 to 5, and in particular 3; Gly designates:

—CH₂—CH(OH)—CH₂—O— or —CH₂—CH(CH₂OH)—O—

Advantageously, the sum of the number of ethylene groups per molecule of compound (B) and the number of hydrogen atoms bonded to the silicon atoms per molecule of compound (A) is at least 4.

Advantageously, compound (A) is added in a quantity such that the molecular ratio between the total quantity of hydrogen atoms bonded to silicon atoms in compound (A) and the total quantity of all of the groups with an ethylene type unsaturated bond in compound (B) is in the range 1/1 to 20/1.

Compound (C) is the catalyst for the cross-linking reaction, and in particular is chloroplatinic acid, chloroplatinic acid-olefin complexes, chloroplatinic acid-alkenylsiloxane complexes, chloroplatinic acid-diketone complexes, platinum black or supported platinum.

The catalyst (C) is preferably added in an amount of 0.1 to 1000 parts by weight, more preferably from 1 to 100 parts by weight, as the pure platinum metal per 1000 parts by weight of the total quantity of compounds (A) and (B).

The polyglycerolated silicone elastomer of the invention is generally mixed with at least one hydrocarbon oil and/or a silicone oil to form a gel. In these gels, the polyglycerolated elastomer is usually in the form of non-spherical particles.

Such elastomers have in particular been described in patent application WO 2004/024798.

Examples of polyglycerolated silicone elastomers that may be used are those sold under the names “KSG-710”, “KSG-810”, “KSG-820”, “KSG-830”, “KSG-840” by the supplier Shin Etsu.

In a preferred embodiment, a polyglycerolated emulsifying silicone elastomer is used in an amount of 25% by weight in polydimethylsiloxane (6 cSt), sold in particular under the name KSG-710 by the supplier Shin Etsu.

In exemplary embodiments of the invention, said silicone elastomers may be present in the composition of the invention in a total quantity of active material of at least more than 0.8% by weight relative to the total weight of the composition, in particular in the range 1% to 4% by weight, preferably 1.5% by weight or more, in particular in the range 1.5% to 2.5% by weight, and more preferably 1.6% by weight or more, in particular in the range 1.6% to 2% by weight.

Other Compounds

In known manner, all of the compositions of the invention may contain one or more adjuvants that are routine in the cosmetic or dermatological fields, gelling agents and/or hydrophilic or lipophilic thickening agents; fillers; moisturizing agents; emollients; hydrophobic or lipophilic active ingredients; free radical scavengers; sequestrating agents; antioxidants; preservatives; alkalinizing or acidifying agents; fragrances; film-forming agents; colorants; and mixtures thereof. The quantities of these various adjuvants are those conventionally used in foundations.

The compositions of the invention may include a pulverulent phase that in particular may comprise one or more fillers and/or pigments.

In a particular embodiment, a composition of the invention may comprise at least 10% by weight, in particular more than 15%, or even more than 20% by weight of a pulverulent phase. It is dispersed in a homogeneous and stabilized form.

In the context of the present invention, the term “pulverulent phase” encompasses all pigment and/or filler type particles as defined below.

The fillers may be present in the emulsion in a quantity in the range 0.1% to 10% by weight relative to the total weight of the emulsion, preferably 0.1% to 7%.

Particular mention may be made of talc, mica, silica, kaolin, starch, boron nitride, calcium carbonate, magnesium carbonate or bicarbonate, microcrystalline cellulose, powders of synthetic polymers such as polyethylene, polyesters, or polyamides such as those sold under the trade name “Nylon”, polytetra/fluoro/ethylene (“Teflon”), and silicone powders.

The colorants may be hydrosoluble colorants of vegetable, mineral or synthetic origin, pigments or nacres.

According a particularly preferred embodiment, a composition of the invention comprises at least one colorant, in particular at least one pigment.

Advantageously, the pigments may be present in a hydrophobic coated form in the emulsion of the invention. More particularly, they may be pigments that have been surface treated with a hydrophobic agent to render them compatible with the fatty phase of the emulsion, in particular so that they are properly wettable by the oils of the fatty phase. Thus, said treated pigments are properly dispersed in the fatty phase.

The pigments intended to be coated may be mineral or organic pigments. Pigments that may be used are metallic oxides such as oxides of iron, in particular those with a yellow, red, brown or black color, titanium dioxides, cerium oxide, zirconium oxide, chromium oxide; manganese violet, Prussian blue, ultramarine blue, ferric blue, bismuth oxychloride, nacre, mica coated with titanium or bismuth oxychloride, colored nacreous pigments such as mica titanium with iron oxides, mica titanium with ferric blue or chromium oxide, mica titanium with an organic pigment of the above-mentioned type, as also nacreous pigments based on bismuth oxychloride, and mixtures thereof.

Preferably, iron oxide or titanium dioxide pigments are used.

The hydrophobic treatment agent may be selected from silicones such as methicones, dimethicones, perfluoroalkylsilanes; fatty acids such as stearic acid; metallic soaps such as aluminum dimyristate, the alkyl salt of hydrogenated tallow glutamate, perfluoroalkyl phosphates, perfluoroalkyl silanes, perfluoroalkyl silazanes, hexafluoropropylene polyoxides, polyorganosiloxanes containing perfluoroalkyl perfluoropolyether groups, amino acids; N-acyl amino acids or their salts; lecithin, isopropyl trisostearyl titanate, and mixtures thereof.

The N-acyl amino acids may comprise an acyl group containing 8 to 22 carbon atoms, such as a 2-ethyl hexanoyl, caproyl, lauroyl, myristoyl, palmitoyl, stearoyl, or cocoyl group. The salts of these compounds may be aluminum, magnesium, calcium, zirconium, zinc, sodium, or potassium salts. The amino acid may, for example, be lysine, glutamic acid, or alanine.

The term “alkyl” as used in the above-mentioned compounds designates an alkyl group containing in the range 1 to 30 carbon atoms, preferably containing in the range 5 to 16 carbon atoms.

Hydrophobic treated pigments are in particular described in the application EP-A-1 086 683.

In a preferred embodiment, a composition of the invention comprises at least one pigment chosen among iron oxides and titanium oxide, in particular iron oxides.

The coated hydrophobic pigments may be present in a quantity in the range 0.5% to 20% by weight relative to the total weight of the composition, preferably in a quantity of at least 5% by weight, in particular in the range 5% to 20% by weight, more particularly from 8% to 20% by weight, and more preferably in the range 8% to 15% by weight.

Similarly, the compositions of the invention may contain active ingredients.

Examples of active ingredients that may be used in the composition of the invention and that may be mentioned are moisturizing agents such as protein hydrolysates and polyols such as glycerin, glycols such as polyethylene glycols, and sugar derivatives thereof; natural extracts; vitamins such as vitamin A (retinol), vitamin E (tocopherol), vitamin C (ascorbic acid), vitamin B5 (panthenol), vitamin B3 (niacinamide), and derivatives of said vitamins (in particular esters) and mixtures thereof; urea; caffeine; salicylic acid and derivatives thereof; alpha-hydroxyacids such as lactic acid or glycolic acid and derivatives thereof; retinoids such as carotenoids and derivatives of vitamin A; sunscreens; extracts from algae, fungi, plants, yeasts and bacteria; enzymes; tightening agents; agents acting on the microcirculation, and mixtures thereof.

The sunscreens (or UV screens) may be selected from organic screens, physical screens and mixtures thereof.

The chemical sunscreens that may be used in the composition of the invention may comprise any UVA or UVB screen that can be used in the cosmetics field.

In particular exemplary embodiments, the composition of the invention may include at least one agent acting on the cutaneous microcirculation.

The active ingredient acting on the cutaneous microcirculation may be used to avoid fading of the tint and/or to improve the appearance of the contour of the eye, in particular to reduce dark rings. It may, for example, be selected from manganese gluconate (Givobio GMn® from the supplier Seppic), extract of lupin (Eclaline® from the supplier Silab), bigarade flower extract (Remoduline® from the supplier Silab), ruscus extract, horse chestnut extract, ivy extract, ginseng extract and sweet clover extract, caffeine, an extract of black tea such as Kombuchka from the supplier Sederma; rutine salts; and an extract of corallina officinalis algae such as that sold in particular by the supplier CODIF; and mixtures thereof.

In particular exemplary embodiments, the composition of the invention may include at least one cooling agent.

A variety of cooling agents are known, as menthol or herbal extracts, and/or mint, Aloe Vera or ginseng essential oils.

The cooling agent may comprise an endothermic organic salt as potassium chloride, mint and derivatives thereof such as menthol, peppermint oil, wintergreen, menthone, menthyl lactate, green mint, mint oil, menthane derivatives such as N-substituted menthane carboxamides, and in particular N-ethyl-p-menthane carboxamide-3,3-(1-menthoxy)-propan-1,2-diol, p-menthane-3,8-diol, menthyl succinate and alkaline earth metal salts thereof, and mixtures thereof, or a nitrogen-containing compound, for example urea.

These cooling agents are generally present in the composition in a quantity in the range 0.01% to 20% by weight, preferably in the range 0.1% to 10% by weight relative to the total weight of said composition.

According to a particular embodiment of the invention, the composition of the invention is different from deodorant and antiperspirant formulations.

In particular, a composition of the invention may advantageously comprise less than 2% by weight of deodorant or antiperspirant actives relative to the total weight of the composition, in particular less than 1% by weight and more preferably is totally free of deodorant or antiperspirant actives.

Said actives that are termed “antiperspirant” or “deodorant” are well known to the skilled person. Antiperspirant or deodorant actives that may be mentioned include active salts such as aluminium or zirconium salts, bacteriostatic agents and bactericidal agents such as quaternary ammonium salts, etc.

According to an other particular embodiment, the composition of the invention is different from a soap composition.

Clearly, the skilled person will be careful to select any adjuvants that are added to the composition of the invention such that the advantageous properties intrinsically attached to the composition of the invention are altered little or not at all by the envisaged addition.

These adjuvants are generally present in the composition in a quantity in the range 0.01% to 20% by weight, preferably in the range 0.1% to 10% by weight relative to the total weight of said composition.

The invention is illustrated in the examples shown below by way of non-limiting illustration of the invention.

Unless otherwise indicated, the values in the examples below are expressed as percentages by weight relative to the total composition weight.

### Example 1

Fluid Foundation Composition (O/W Emulsion)

| Ingredients | % by weight |
|---|---|
| Pure sodium hydroxide | 0.30 |
| Purified smectite | 0.50 |
| Dicaprylyl ether (Cetiol OE ® from Cognis) | 17 |
| Titanium oxide (untreated anatase) (CI: | 10.04 |
| 77891) | |
| Black, red and yellow iron oxides (CI: | 2.96 |
| 77891, 77499 and 77492) | |
| Glycerin of vegetable origin, including palm | 6 |
| Deionized water | 40 |
| Ethyl alcohol, 96°, non denatured | 6 |
| Mixture of glyceryl mono/distearate (36/64)/ | 6 |
| potassium stearate (Tegin Pellets ® from | |
| Evonik Goldschmidt) | |
| Preservatives | 1 |
| Glyceryl mono/distearate | 1 |
| Fatty acids of vegetable origin (principally | 1.5 |
| stearic acid) | |
| D-alpha-tocopherol (natural vitamin E) | 0.2 |
| Silica micro-beads (granulometry from 3 μm | 2 |
| to 15 μm) | |
| Liquid jojoba wax | 5.5 |
| Total | 100 |

Operating Method:

The aqueous phase was prepared without the ethyl alcohol until the preservatives had dissolved.

The fatty phase was prepared with the surfactants in a 1 L [liter] beaker then heated on a hotplate to 80° C. to melt the wax. The smectite was then added and the mixture was allowed to swell for 15 min with stirring on the hotplate.

A pigment paste was prepared by dispersing the pigments in the oil. The mixture was passed through a three cylinder mill 3 times.

The fillers were then added and allowed to disperse for 15 min in the fatty phase in a Moritz homogenizer.

Emulsification was then carried out by pouring the aqueous phase into the fatty phase. It was allowed to emulsify for 10 min.

The alcohol was then introduced at ambient temperature. Next, the solution was degassed in a Rayneri mixer using a propeller.

This composition had a viscosity of 1.3 Pa·s.

### Example 2

Fluid Foundation Composition (W/O Emulsion)

| | % by |
|---|---|
| Ingredients | weight |
| Sequestrating agents | 0.2 |
| Sodium chloride | 1 |
| Titanium oxide and C₉-C₁₅ fluoroalcohol | 9.28 |
| phosphate | |
| Iron oxides and C₉-C₁₅ fluoroalcohol | 1.72 |
| phosphate | |
| Preservatives | 1 |
| Dimethicone | 2.50 |
| PEG-9 polydimethylsiloxyethyldimethicone | 2 |
| and PEG-9 (KF6028 from Shin Etsu) | |
| Dimethicone and cross-linked dimethicone/ | 7 |
| polyglycerin-3 polymer (KSG 710 from Shin | |
| Etsu) | |
| Ethanol | 5 |
| Water | 42.80 |
| Glycerin | 5 |
| Cyclopentasiloxane | Qsp 100 |

Operating Method:

A pigment paste was prepared by dispersing the pigments in the silicone then passing the mixture through the three cylinder mill three times.

Further, the non-volatile oils and the KF6028 silicone surfactant were mixed with a Moritz homogenizer at 820° C., adding the preservatives. Heating was halted and when the mixture temperature dropped below 50° C., the remainder of the cyclopentasiloxane was added.

The emulsifying silicone elastomer (KSG 710) was then added and the mixture was stirred gently in a turbine for 10 minutes.

At the same time, the aqueous phase was prepared by mixing the preservatives, glycerin, water with stirring to 70° C. and then allowing the mixture to cool. The alcohol was then added.

Emulsification was carried out by stirring at the maximum rate for 10 minutes.

This composition had a viscosity of 3 Pa·s.

Results

Application of this composition with an applicator device as described in FIGS. 1 to 3 was compared with the application with a conventional sponge.

Application was carried out using an applicator device over half of the face, the other half being made up using a conventional sponge.

Said sponge and applicator device were weighted before and after the application over each half of the face so that the same quantity (0.20g) of composition is applied over each half of the face.

The application sensations (fresh effect, softness, slippery effect), makeup result (uniformity, coverage) were evaluated by the user; symbols “+” to “+++” being attributed according to satisfaction of the user.

In terms of the coverage property, it is appreciated visually after application according to change in color of the skin, masking of relief imperfections of the skin, etc.

A table 1 is reported below, giving the noted results for the two applications.

TABLE 1

| | | Application with the |
|---|---|---|
| | Application with a | applicator device of the |
| Evaluation | conventional sponge | invention |
| Fresh effect at the | +++ | +++ |
| first passage | | |
| Fresh effect over time | + | +++ |
| Uniformity of the | + | ++ |
| deposit | | |
| Uniformity at the end | ++ | +++ |
| of the makeup | | |
| Application softness | + | ++ |
| Application slippery | ++ | +++ |
| effect | | |
| Coverage property | + | ++ |

In particular, it is much more difficult to get a makeup with satisfactory uniformity by applying the composition with a conventional sponge in comparison with the application with the applicator device of the invention.

In terms of the makeup results, the makeup resulting from application with the applicator device of the invention presents a better uniformity.

In term of fresh effect, the application with the applicator device of the invention maintains the freshness over a long time.

### Example 3

Fluid Foundation Composition (W/O Emulsion)

| Ingredients | % by weight |
|---|---|
| Magnesium sulfate | 0.7 |
| Magnesium gluconate | 0.1 |
| Expanded micro-spheres of vinylidene chloride/acrylonitrile/PMMA in | 0.5 |
| isobutane (EXPANCEL 551 DE 20 D60 from Expancel) | |
| Iron and titanium oxide (sold under the reference PF 5 by DAITO KASEI | 14 |
| KOGYO) | |
| Preservative | 1 |
| Mixture of acetylated ethylene glycol stearate, glyceryl tri-stearate (sold | 0.5 |
| under the reference UNITWIX by United Guardian) | |
| Nylon-12 | 3 |
| Poly methylcetyl dimethylsiloxane, oxyethylenated (20/75/5-viscosity: | 3 |
| 3000 cSt) sold under the reference ABIL EM 90 by EVONIK | |
| GOLDSCHMIDT | |
| Microbeads of methylsilsesquioxane resin (granulometry: 4.5 μm) (sold | 2 |
| under the reference TOSPEARL 145 A by MOMENTIVE | |
| PERFORMANCE MATERIALS) | |
| Mixture of polydiphenyl dimethylsiloxane (gum-viscosity >1000000 cSt- | 4 |
| MPt: 600000) and of cyclopentadimethylsiloxane (15/85) (sold under the | |
| reference SILBIONE 71634 by BLUESTAR) | |
| Mixture of oxyethylenated/oxypropylenated poly dimethylsiloxane (18 | 2.55 |
| OE/18 OP), cyclopentadimethylsiloxane and water (10/88/2) (sold under | |
| the reference DOW CORNING 5225C FORMULATION AID by DOW | |
| CORNING) | |
| Polyglycerolated diisostearate (3 moles) | 0.5 |
| Deionized water | 31 |
| Guar gum, 2-hydroxypropyl ether | 0.1 |
| Propylene glycol | 3 |
| Glycerol | 5 |
| Isododecane | 7 |
| Cyclopenta dimethylsiloxane sold under the reference XIAMETER | Qsp 100 |
| PMX-0245 CYCLOSILOXANE by DOW CORNING | |

A pigment paste was prepared by dispersing the pigments in silicone, then the mixture was passed through the three cylinder mill 3 times.

Next, the non-volatile oils, the surfactants and the preservatives were mixed at 75° C. on a hotplate with magnetic stirring. The silicone oils were then added and dispersed thoroughly. The mixture was stirred with a Moritz homogenizer with a large turbine.

Heating was halted and at about 30° C., the milled pigment prepared as above was added. The fillers were then added one by one, followed by the isododecane.

At the same time, the aqueous phase was prepared by mixing the preservatives, glycerin, water with stirring using a Rayneri using a high vortex, then the hydroxypropyl guar was added and the gel was allowed to swell. The salts were then added with stirring.

The emulsion was formed by stirring at maximum rate for 10 minutes.

The Expancel 551 was added to the emulsion, stirring in the Rayneri with a propeller.

This composition had a viscosity of 1.4 Pa·s.

Results:

The application sensations (fresh effect, softness, slippery effect), makeup result (uniformity, coverage) were evaluated by the user when the composition is applied with an applicator device as described in FIGS. 1 to 3, compared with the application with a conventional sponge, according to the evaluation protocol described above for the composition of Example 2.

A table 2 is reported below, giving the noted results for the two applications.

TABLE 2

| | | Application with the |
|---|---|---|
| | Application with a | applicator device of the |
| Evaluation | conventional sponge | invention |
| Fresh effect at the | + | +++ |
| first passage | | |
| Fresh effect over time | + | +++ |
| Uniformity of the | + | +++ |
| deposit | | |
| Uniformity at the end | + | +++ |
| of the makeup | | |
| Application softness | + | +++ |
| Application slippery | + | +++ |
| effect | | |
| Coverage property | + | +++ |

It is difficult to apply this composition with a conventional sponge as the formulation dries quickly and lacks of slippery effect.

In terms of the makeup results, the makeup resulting from application with the applicator device of the invention presents a better uniformity.

In term of fresh effect, the application with the applicator device of the invention maintains the freshness over a long time.

Applying the compositions of Examples 1 to 3 to the face with an applicator device as described in FIGS. 1 to 3 bestowed a great sensation of freshness. This fresh feel was much greater than that felt with application of the same compositions using the fingers.

The applicator device also had the advantage of not soiling the fingers with foundation.

In terms of the makeup result, the makeup obtained was uniform, thin, uniform and smooth, producing a discrete and natural result.

Finally, the user appreciated the light massage produced by stroking when applying said composition with said device.

1. A kit for making up and/or for care of keratinous material comprising:
a container containing at least one cosmetic composition, the at least one cosmetic composition being a water-in-oil, oil-in-water or multiple emulsion, and comprising at least 30% by weight of aqueous phase relative to the total weight of said composition and having a viscosity measured at 25° C. and at a shear rate of 200 min⁻¹ of more than 0.8 Pa·s, said composition comprising at least one colorant; and
an applicator device configured to apply the composition, the applicator device comprising an applicator member having an application surface configured to turn about at least one axis or center of rotation in response to being moved into engagement with the keratinous material.

2. The kit according to claim 1, wherein the composition includes a range of 30% to 70% by weight of aqueous phase, relative to the total weight of said composition.

3. The kit according to claim 1, wherein the composition includes a range of 20% to 40% by weight of fatty phase, relative to the total weight of said composition.

4. The kit according to claim 1, wherein the composition is a fluid.

5. The kit according to claim 1, wherein the composition has a viscosity, measured at 25° C. and at a shear rate of 200 min⁻¹, of more than 0.8 Pa·s and less than or equal to 4 Pa·s.

6. The kit according to claim 1, wherein the aqueous phase includes one or more C₂ to C₈ lower mono-alcohols.

7. The kit according to claim 1, wherein the composition includes at least one surfactant selected from amphoteric, anionic, cationic and non-ionic surfactants, used alone or as a mixture.

8. The kit according to claim 7, wherein the at least one surfactant is present in a total quantity ranging from 3% to 20% by weight relative to the total weight of the composition.

9. The kit according to claim 1, wherein the composition includes at least one silicone surfactant selected from dimethicone copolyols.

10. The kit according to claim 1, wherein the composition is chosen from a blusher, an eye shadow, a foundation, a concealer, a lipstick, a body makeup product, a face care product, a body care product and a sunscreen.

11. The kit according to claim 1, wherein the composition is a foundation.

12. The kit according to claim 1, wherein the applicator member is a roller.

13. The kit according to claim 12 wherein the roller has an application surface having material configured to retain the composition, said material chosen from an elastomeric material, a foam with open or closed cells, a flocked membrane, a sintered material, a woven material and a nonwoven material.

14. A method of making-up and/or caring for a keratinous material, the method comprising:
loading an applicator member with a quantity of a composition with the applicator member having an application surface configured to turn about an axis or a center of rotation in response to being moved into engagement with keratinous material; and
applying the composition to keratinous material with said applicator member, said composition being a water-in-oil, oil-in-water or multiple emulsion, and comprising at least 30% by weight of aqueous phase relative to its total weight, and having viscosity measured at 25° C. and at a shear rate of 200 min⁻¹ of strictly more than 0.8 Pa·s, and said composition comprising at least one colorant.

15. The method according to claim 14, wherein the composition includes a range of 30% to 70% by weight of aqueous phase, relative to the total weight of said composition.

16. The kit according to claim 2, wherein the composition includes at least 32% by weight of aqueous phase, relative to the total weight of said composition.

17. The kit according to claim 2, wherein the composition includes a range of 35% to 45% by weight of aqueous phase, relative to the total weight of said composition.

18. The kit according to claim 3, wherein the composition includes a range of 22% to 30% by weight of fatty phase, relative to the total weight of said composition.

19. The kit according to claim 3, wherein the composition includes a range of 22% to 26% by weight of fatty phase, relative to the total weight of said composition.

20. The kit according to claim 5, wherein the composition has a viscosity ranging from 0.9 Pa·s to 3.8 Pa·s.

21. The kit according to claim 5, wherein the composition has a viscosity ranging from 1 Pa·s to 3.5 Pa·s.

22. The kit according to claim 6, wherein the one or more mono-alcohols are present in a quantity of at least 2% by weight relative to the total weight of the composition.

23. The kit according to claim 8, wherein the at least one surfactant is present in a total quantity ranging from 3.5% to 15% by weight relative to the total weight of the composition.

24. The kit according to claim 8, wherein the at least one surfactant is present in a total quantity of more than 5% by weight relative to the total weight of the composition.

25. The kit according to claim 8, wherein the at least one surfactant is present in a total quantity ranging from 5.1% to 10% by weight relative to the total weight of the composition.

26. The method according to claim 14, wherein the composition comprises a range of 20% to 40% by weight of fatty phase, relative to the total weight of said composition.

27. The method according to claim 14, wherein the composition is a fluid.

28. The method according to claim 14, wherein the composition has a viscosity, measured at 25° C. and at a shear rate of 200 min⁻¹, of more than 0.8 Pa·s and less than or equal to 4 Pa·s.

29. The method according to claim 14, wherein the aqueous phase comprises one or more C₂ to C₈ lower mono-alcohols.

30. The method according to claim 14, wherein the composition comprises at least one surfactant selected from amphoteric, anionic, cationic and non-ionic surfactants, used alone or as a mixture.

31. The method according to claim 30, wherein the at least one surfactant is present in a total quantity ranging from 3% to 20% by weight relative to the total weight of the composition.

32. The method according to claim 14, wherein the composition comprises at least one silicone surfactant selected from dimethicone copolyols.

33. The method according to claim 14, wherein the composition is chosen from a blusher, an eye shadow, a foundation, a concealer, a lipstick, a body makeup product, a face care product, a body care product and a sunscreen.

34. The method according to claim 14, wherein the composition is a foundation.

35. The method according to claim 14, wherein the applicator member is a roller.

36. The method according to claim 35, wherein the roller has an application surface having material configured to retain the composition, said material chosen from an elastomeric material, a foam with open or closed cells, a flocked membrane, a sintered material, a woven material and a nonwoven material.
